(19) United States
(12) Patent Application Publication
(10) Pub. No.: US 2002/0095257 A1
(43) Pub. Date: Jul. 18, 2002

(54) Method and system for detection by raman measurements of bimolecular markers in the vitreous humor

(76) Inventors: Richard B. Rosen, New York, NY (US); Erik F. Kruger, Cambridge, MA (US); Alvin Katz, Bronx, NY (US); Robert R. Alfano, Bronx, NY (US)

Correspondence Address: LADAS & PARRY, 26 WEST 61ST STREET, NEW YORK, NY, 10023, US

(21) Appl. No.: 09/817,760

(22) Filed: Mar. 26, 2001

Related U.S. Application Data
(60) Provisional application No. 60/192,280, filed on Mar. 27, 2000.

(51) Int. Cl. G06F 19/00; G01N 33/48; G01N 33/50; A61B 3/10

(52) U.S. Cl. 702/19; 351/205

(57) ABSTRACT

A system to detect eye disease in which monochromatic laser light is directed into the vitreous humor after passing through the front of the eye. A very sensitive detection system then detects the light scattered from the vitreous humor as it exits the eye. The light is scattered at a wavelength different from that of the laser in a manner known as Raman scattering. The wavelength of the Raman scattered photons are shifted by vibrational modes of the molecules, and this shift is a characteristic feature of the molecules interacting with the light. In this way, the Raman scattered light is essentially imprinted with a fingerprint of relevant molecules. As it exits the eye, this Raman scattered light can be separated from other types of scattered light and then routed to a detection system, wherein the results are calibrated against actual standards for the particular vitreous substances being analyzed. An optic arrangement tightly focuses the laser into the vitreous humor and thereby reduces the spectral fingerprints or noise from proteins and other molecules normally present in the lens, cornea, retina and other eye components.

### FIELD OF THE INVENTION

The present invention relates to methods and apparatus for measuring levels of chemical compounds located in the anatomy of the eye, and more specifically to methods and apparatus for measuring such compounds in the vitreous humor of the eye for assessing the risk of suffering diseases of the eye and monitoring the progression of these diseases as they are treated.

The present invention relates particularly to methods and apparatus for assessing the presence and activity of extrinsic biomolecules which indicate a variety of disease processes. The present invention provides a non-invasive, rapid, and objective determination of levels of these bimolecules.

### BACKGROUND

In a technique referred to as laser Raman spectroscopy, monochromatic laser light is directed onto a particular material to be tested. A very sensitive detection system then detects light returning, or scattered, from the material. This majority of the light returning from the material is scattered elastically (at the same wavelength of the original laser light) in a manner known as Rayleigh scattering. A very small fraction of the light returning from the material is scattered inelastically at a wavelength different from that of the original projected laser light in a manner known as Raman scattering. The Raman scattered light is then separated from the Rayleigh scattered light with the use of filters, optical gratings, prisms, and other wavelength selection techniques. The energy difference between Raman and Rayleigh scattered light, thereof, of various molecules in the material being evaluated. Each of the peaks in the resulting Raman spectrum corresponds to a particular Raman active vibration of a molecule or a component thereof. The Raman energy “shift” is independent of the wavelength of the directed laser light. That is, the energy difference corresponding to the elastically and inelastically scattered light for a particular material remains constant for that material.

The characteristic results from Raman scattering can be used to locate, identify and quantitate concentrations of a material. The absolute intensities of the resulting Raman peaks are directly related to the concentration of the Raman-active molecules in the material. Either spectral changes in a patient over time, or differences in spectrum between a patient and known Raman standards from the normal eye shows the onset of disease.

Vision, or sense of sight, is one's ability to perceive the form, color, size, movement, and distance of objects, by way of a complex anatomy generally termed the eye. Vision occurs when light passes through the eye and is absorbed by the photo receptors, the sensitive cells of the retina in the back of the eye. Specifically, light enters the cornea of the eye, passes through the pupil, the lens, and the vitreous humor, and finally falls upon the retina. Human vision is particularly sensitive to light in the visible spectrum, which is from approximately 380-720 nanometers in wavelengths.

The majority of the space behind the lens of the eye is filled with a substance called the vitreous humor. The bulk of the vitreous humor is water (approximately 98°) with a small amount of collagen, hyaluronic acid, and several other small molecules. The vitreous humor performs a variety of structural and biochemical functions. A diagram showing the structure of the eye is shown in FIG. 1 (1).

The eye, specifically the lens and cornea, is rich in a variety of proteins, many of which exhibit Raman emission bands (2-4). Accurate measurements of molecules in the vitreous will require that the desired emission signals be separated from the background emissions. This may be accomplished either optically, by the use of data processing algorithms, or a combination of the two. The current invention includes optical designs which can restrict data collection to specific regions of the eye thereby eliminating unwanted signals or noise from other parts of the eye.

Glaucoma is the second most common cause of legal blindness in the United States. It is the leading cause of blindness for African-Americans. Furthermore, about 2 million Americans have glaucoma, but only half of them are aware of it. The earlier therapy is initiated for this blinding disorder, the better the chance of saving useful vision. Similarly, diabetic retinopathy is a potentially blinding complication of diabetes that damages the retina. It affects half of all Americans diagnosed with diabetes. With timely treatment, 90 percent of those with advanced diabetic retinopathy can be saved from going blind. The benefits derived from improved screening, as well as in diagnosis and treatment, of these potentially blinding disorders would therefore have a major impact on these highly prevalent diseases, as well as several others. It has been demonstrated that L-glutamate plays a pivotal role in the neurotoxic process termed excitotoxicity. This critical process is the final common pathway for a variety of pathophysiologic insults that initiate cell death. This process has been shown to be operative in several ocular and non-ocular diseases. Ocular diseases in which this process is at least, in part implicated, include glaucoma, diabetic retinopathy, retinal detachment, and several retinal degenerative disorders. In glaucoma, it would be a significant advance to be able to monitor biochemical changes in the vitreous humor, such as increased levels of glutamate, as an objective reflection of neuronal death before the disease process leads to the actual, irreversible physical changes in the ocular structures that are observable clinically. In this manner, treatment could be initiated at a significantly earlier stage in the disease process thus helping to preserve vision. Currently, there are no techniques to non-invasively evaluate the levels of glutamate in the vitreous humor.

Glutamate is the principal excitatory neurotransmitter in the central nervous system, including the eye. When released by nerve cells in physiologic quantities, it is involved in a number of normal processes. However, with nerve cell death from a variety of causes, these cells rupture and release their internal stores of glutamate into the surrounding tissue. This overstimulates surrounding nervous cells initiating a process that results in their demise, rupture, and the release of even more glutamate. This process has been termed glutamate-mediated neurotoxicity or excitotoxicity. Glutamate-mediated neurotoxicity has been shown to play a role in a variety of ocular diseases including glaucoma, diabetic retinopathy, and retinal detachment. Taken together, these diseases represent a major source of visual disability in the United States. When these disease processes are present, it has been shown that there are increased levels of glutamate in the structurally adjacent vitreous humor, probably due to spill over from the glutamate being elaborated in the retina in large quantities. It is currently only possible to detect the levels of glutamate in the vitreous humor by an invasive technique. This technique measures vitreous humor glutamate levels from eyes undergoing surgery by removing a small amount of vitreous humor and analyzing it by way of conventional biochemical means such as high performance liquid chromatography (HPLC). However, this technique clearly suffers from the absence of any value for use in connection with diagnosing and monitoring non-surgical patients (the majority) over an extended period of time. This technique has been purely a research technique and is not in routine use because of its lack of practicality. There is currently no technique for the non-invasive measurement of glutamate in the vitreous humor of human eyes.

### SUMMARY OF THE INVENTION

An object of the invention is to provide a system for performing in situ examination of the eye of a patient for detecting the presence and changes in the vitreous humor which are evidence of various eye diseases.

A further object of the invention is to provide such a system in which collection of information from the eye is restricted to specific regions to eliminate unwanted signals or noise from other parts of the eye.

The invention comprises a system for performing in situ examination of the eye of a patient for detecting the presence and changes in biomolecular indicators in the vitreous humor which are evidence of various eye diseases, the system comprising a monochromatic light source for directing a monochromatic light beam through the front of the eye onto the vitreous humor, a detector for detecting scattered light of the laser light beam, a spectroscope for measuring by Raman spetroscopy shift of the scattered light due to the presence of any indicators in the vitreous humor which are evidence of corresponding eye diseases, a diagnostic unit for correlating the shift of the scattered light to determine eye disease, the system being constructed and arranged to minimize noise interference produced outside the vitreous humor.

In accordance with a feature of the invention the monochromatic light source comprises a laser generator.

In accordance with a further feature of the invention, a wavelength selective optical device to isolate and detect the Raman signals.

In accordance with a further feature of the invention, said detector comprises a photodetector for converting light signals to electrical signals.

In accordance with a further feature of the invention, a lens system is provided for expanding said light beam and focusing it into the vitreous humor.

In accordance with a further feature of the invention, optical fibers are provided for delivering the monochromatic light beam to the eye and for collecting the Raman scattered light and filters in the light beam delivering fibers to eliminate fluorescence and Raman light generated in the fibers.

In accordance with a further feature of the invention, further filters are provided in the collecting fibers to eliminate excitation light and other background light sources.

In accordance with a further feature of the invention, that in other to minimize noise interference produced as Raman signals in the lens and cornea said system comprises a lens system for expanding the monodynamic light beam at the lens and focusing the light beam in the vitreous humor.

In accordance with a further feature of the invention, a monitoring system is provided for monitoring changes in Raman spectra from the patient over time to detect onset of disease.

In accordance with a further feature of the invention, the system further comprises a monitoring system for monitoring changes in Raman spectra from the patient over time to measure progression of disease.

In accordance with a further feature of the invention, the system further comprises a device to compare and calculate differences in Raman spectra between a normal eye and the eye of the subject patient wherein said device locates and identifies the differences to track changes in the biomolecules, said device including a detector including signal processing algorithms to enhance measured changes.

In accordance with a further feature of the invention, the system includes a diagnostic unit having cure integration and differentiation means to enhance detection of disease, said diagnostic unit including a system of background subtraction by polynomial, fitting to enhance identification of disease. The diagnostic unit can include a device to carry out Fourier analysis to determine differences in spectra to enhance identification of disease and carry out Fourier filtering to determine differences in spectra to enhance identification of disease.

In accordance with a further feature of the invention, the detector can detect MSG or ascorbic acid.

### BRIEF DESCRIPTION OF THE FIGURES OF THE DRAWING

FIG. 1 is a diagrammatic illustration of the structure of the eye.

FIG. 2 is a graphical illustration of the Raman spectrum of a whole porcine eye.

FIG. 3 is a graphical illustration of the Raman spectrum of the cup of the porcine eye with the lens and cornea removed.

FIG. 4 is a diagrammatic illustration of an embodiment of the apparatus of the invention.

FIG. 5a shows the apparatus with a light beam focused on the retina for focusing the incoming light beam in the vitreous humor.

FIG. 5b shows a lens system for focusing the incoming light beam in the vitreous humor.

FIG. 6 is a graphical illustration of Raman spectra of MSG in aqueous solution.

FIG. 7 is a graphical illustration of Raman spectra of MSG powder.

FIG. 8 is a graphical illustration of a porcine eye injected with MSG, the excitation laser beam being focused on the ocular lens and the Raman bands being masked by the signals from the lens and cornea.

FIG. 9 is a graphical illustration of the Raman spectrum of the porcine eye of FIG. 8 in which the excitation laser beam is focused in the vitreous humor whereby the Raman emission from the lens and cornea is substantially reduced and the MSG signals are evident at 1079, 1344 and 1415 cm⁻¹.

FIG. 10 is a graphical illustration of the Raman spectra of the porcine eye injected with MSG after subtraction of background from the spectrum in FIG. 9.

FIG. 11 is a graphical illustration of the Raman spectrum from the porcine cup immediately after MSG injection and 15 minutes after injection, the MSG not being injected directly into the optical path and therefore taking several minutes to diffuse through the vitreous.

FIG. 12 is a graphical illustration showing the difference spectrum from the porcine eye cup 15 minutes and immediately following injection with MSG, the background being subtracted from the individual spectra using polynomial approximation prior to taking the difference.

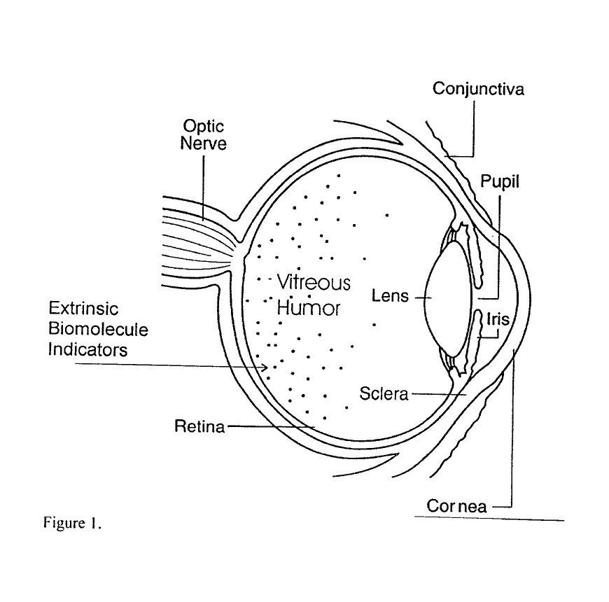

### DETAILED DESCRIPTION OF THE INVENTION

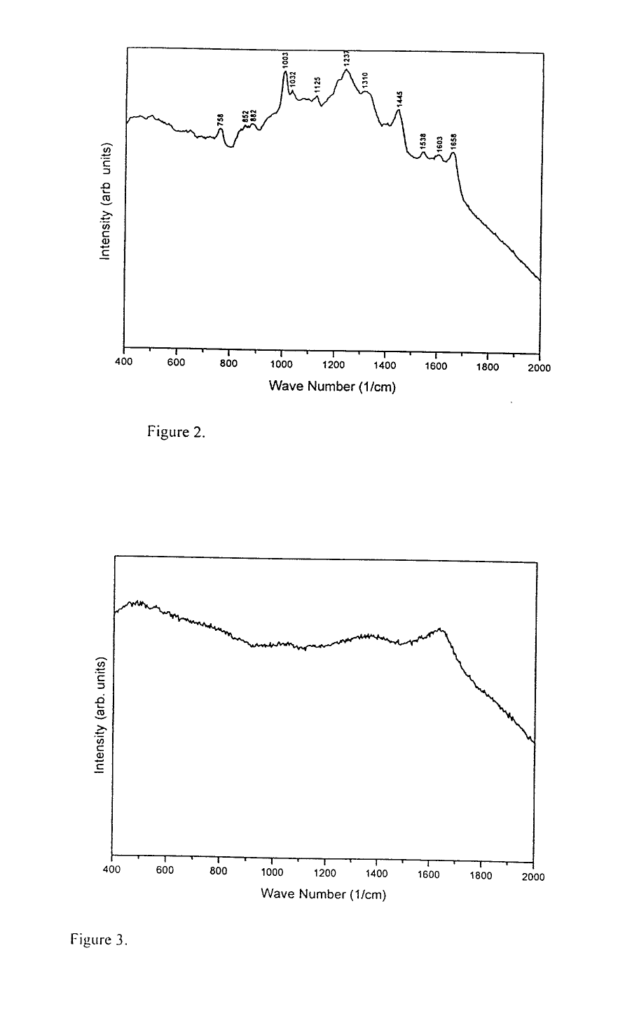

In order to accurately detect low concentrations of extrinsic biomolecules in the eye, it is necessary to eliminate signals generated by naturally occurring molecules in the eye, specifically the lens, cornea and retina. The Raman spectrum of the eye has a complicated structure with many different Raman emissions bands from the different proteins present in the eye. The Raman spectrum from an ex vivo whole porcine eye is shown in FIG. 2. The Raman spectrum of a porcine eye cup (lens and cornea removed) is shown in FIG. 3. This spectrum does not exhibit much of the Raman structure seen in the whole eye. The broad emission peak around 1650 cm⁻¹ is probably an O—H stretching mode of water, of which the vitreous is 98%. These spectra indicate that the major signal contributions are generated in the lens and cornea.

We teach an optical geometry which was developed as part of this invention which would allow collection of signals generated in the vitreous, while simultaneously reducing or eliminating Raman signals generated in the lens, cornea, sclera and retina. The basic principle behind this optical system design is that the use of a large excitation beam diameter coupled with a tight focus gives a very short depth of field. Combined with the use of a small aperture in the collection optics, only signals generated near the focal point of the excitation beam will be coupled into the detection system. In this system, the optics are designed such that the excitation spot size in the vitreous is small, while at the lens and retina, the spot size is large. This design has the added benefit of reduced risk of damage to the retina since the intensity at the retina will be smaller compared to a system with equivalent optical power but with a more collimated excitation laser.

### Optical Design

The lens and cornea of the contain many proteins with distinct Raman spectra. The energy shifts from some of these Raman bands may overlap, and therefore mask the Raman signals from the molecules to be detected, since they generally will be present at lower concentrations than native proteins. The Raman spectra from whole porcine eyes were acquired and a typical spectrum is shown in FIG. 2. The spectrum consists a multiple Raman emission bands and a broad background. Table 1 lists the observed Raman bands, and some suggested assignments (5-7). The background is a combination of long wavelength fluorescence from the eye and stray laser light. The Raman spectrum from the eye cup (eye with cornea and lens removed) was also acquired and is shown in FIG. 3. Most of the Raman structure visible in FIG. 2 is not observed in FIG. 3, indicating that the Raman signal is generated predominantly in the lens and cornea. In FIG. 3, the only Raman contribution observes is the water band around 1600 cm¹. These spectra were acquired with the excitation laser entering the eye through the pupil and being weakly focused in the vitreous.

TABLE 1
RAMAN BANDS OF WHOLE PORCINE
EYES AND SUGGESTED ASSIGNMENTS

| Raman shift | | Raman Shift | |
|---|---|---|---|
| (1/cm) | Assignment | (1/cm) | Assignment |
| 758 | Tryptophan | 1137 | C—N stretching |
| | | | of proteins |
| 797 | | 1251 | Amide III |
| 815 | | 1316 | Collagen twisting |
| | | | mode |
| 834 | | 1350 | |
| 871 | C—C stretch | 1454 | CH₂ Bending |
| | hydroxyproline | | |
| 1007 | Phenylalanine | 1610 | C═C bending |
| | | | of tryptophan |
| 1040 | | 1665 | Amide I |
| 1081 | C—N stretching | | |
| | of protein | | |

As part of this invention's teachings, optical geometries are described with can significantly reduce or eliminate collection of signals generated in the cornea, lens or retina, while collecting signals from the vitreous. This geometric design is based on the use of a large beam size at the lens and retina, combined with a tight focus in the vitreous. An aperture in the image plane of the collection optics, blocks the signals not generated at the focus of the excitation laser (in the vitreous) and prevents these signals from entering the spectrometer and being incident on the detector as noise. The aperture can either be a small diameter collection fiber, or in the case of free space, a pin hole.

FIG. 3 depicts an apparatus for detecting MSG in the vitreous humor of an eye. In this apparatus, monochromatic light is generated by a laser and delivered to the eye by an optical fiber. A second fiber, positioned next to the excitation fiber collects the Raman light and delivers it to a spectrometer for wavelength differentiation and recording. A narrow band filter on the excitation fiber is used to block fluorescence and/or Raman generated in the excitation fiber from being scattered into the collection fiber (8). A notch filter is used in the collection fiber to block the excitation laser light from the detector. The excitation beam, upon exiting the fiber is allowed to expand to a diameter larger than the pupil diameter. A short focal length lens focuses the laser beam to a focal point inside the vitreous. The lens focal length is selected such that the beam diameter at the lens is equal to the diameter of the dilated pupil. In this optical geometry, only Raman signal generated at the focal point, in the vitreous, is effectively collected by the collection fiber.

Other optical constructions which also reduce or eliminate the Raman signal from the lens and cornea, while collecting Raman signal from the vitreous would be within the scope of this invention. Examples of such a system using free space optics and apertures is depicted in FIG. 5. In this system (bottom image in FIG. 5) the excitation laser beam is expanded, collimated and directed towards the eye to be examined. A short focal length lens, in conjunction with the ocular lens, brings the beam to a focus in the vitreous. The precise location inside the vitreous can be controlled by the position of the focusing lens relative to the ocular lens. The relative position of the lens can be controlled by mounting the lens on a frame, such as the type used in a slit lamp. The frame serves to hold the patient's head in a fixed position, thus the lens and eye are fixed relative to each other.

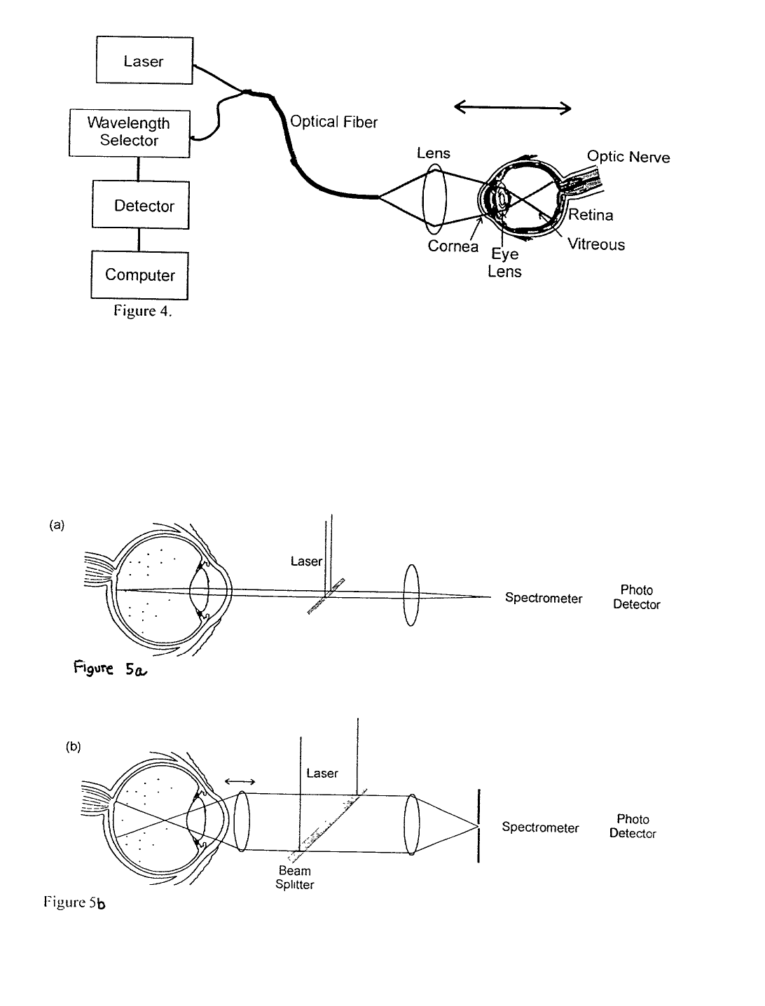

Other means for generating light are within the scope of the present invention, including, but not limited to light sources that generate monochromatic light, and any other light projection system. It should also be understood that the present intention is not limited to generated light at any particular wavelength. For example, other wavelengths of generated light would be effective with the apparatus of the present invention. The generated light is preferably directed to the subject eye via a light delivery system. In FIG. 4, this is achieved in a “bench” set up via simple optical elements. It should be appreciated, however, that various delivery means for directing the generated light would be within the scope of the present invention. For example, one preferred delivery means for directing generated light in the clinical setting is a slit lamp. Other preferred delivery means include, but are not limited to direct ophthalmoscopes, indirect ophthalmoscopes, and mirrors. Alternatively, the delivery means for directing generated light may incorporate a small beam scanned across the vitreous in a manner analogous to the method used in the scanning laser ophthalmoscope, known to those skilled in the art. The returning light scattered from the vitreous of the eye is emitted through the pupil, where it is then collected via a light collection system. In FIG. 4 this is achieved by an optical fiber, filter and lens. It should also be appreciated that other light collection means for collecting the returning light scattered from the vitreous would be within the scope of the present invention. Such light collection means includes optical fibers, lens, mirrors, and combinations thereof. The scattered light is then routed to a spectrally selective system which selects only the Raman scattered light and rejects the Rayleigh scattered light, such that the Raman signals maybe analyzed absent of interference from Rayleigh signals. In FIG. 4, this is achieved by a grating. It should be understood that any spectrally selective means for filtering scattered light which is able to filter elastically scattered light from inelastically scattered light would be within the scope of the present invention. Examples include, but are not limited to, ruled gratings, holographic gratings, holographic filters, prisms, dielectrics coatings, or combinations thereof. After the scattered light is spectrally selected, it is channeled to a light detection system which measures the intensity of the scattered light as a function of wavelengths in the region of Raman peaks characteristic of vitreous MSG. In FIG. 4, this is achieved with a CCD. In alternative embodiments, other light detection means for measuring the intensity of the scattered light such as a photo multiplier, or any other sensitive photo detector such as a photo diode, would also be within the scope of the present invention, the light detection system converts the scattered light signal to an electrical signal, and so that it may be displayed visually such as on a computer monitor or other similar screen. It should be understood, however, that the light detection system may convert the scattered light signal into a format for numerical, digital, or other form of detection.

### Detection of Monosodium-1-glutamate (MSG)

The present invention was applied to detecting MSG in ex vivo porcine eyes.

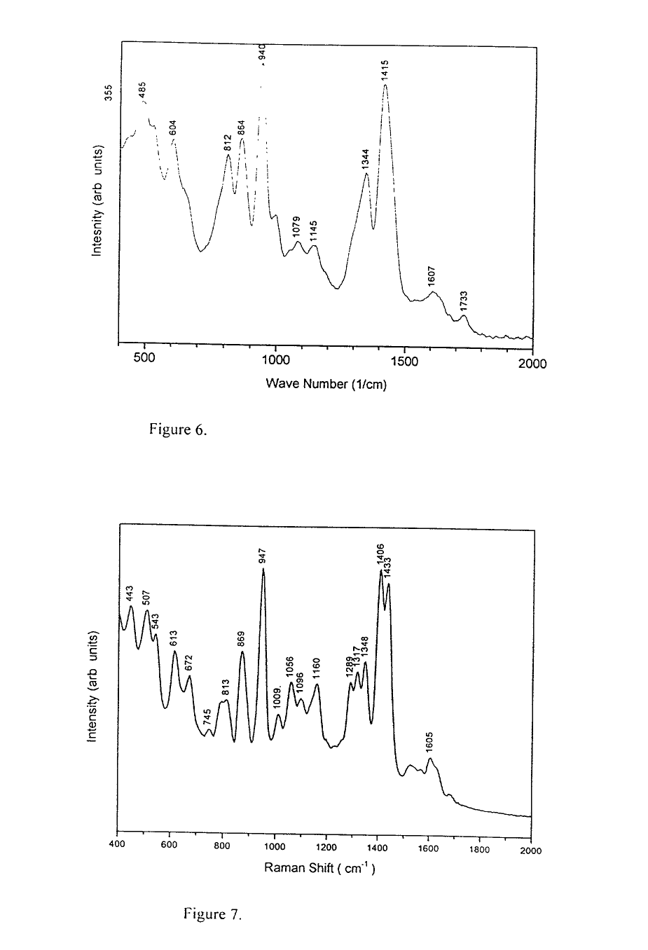

The Raman spectra for MSG in aqueous solution and in powder form was acquired and the energy shifts of the Raman emission bands identified. The Raman spectrum from MSG in aqueous solution is shown in FIG. 6, and in power form in FIG. 7. The five most intense Raman peaks from the MSG solution were at 812, 864, 940, 1344 and 1415 cm⁻. Additional Raman emission lines were also identified and can be observed in FIG. 6.

These characteristic Raman bands can be used to locate, identify and quantitate concentrations of MSG. The absolute intensities of these Raman peaks are directly related to the concentration of MSG present in the sample. The Raman shifts from MSG are summarized in Table 2.

```
TABLE 2
MAIN RAMAN BANDS OBSERVED
IN AQUEOUS SOLUTION OF MSG
Raman Shift
(1/cm)
  812
  864
  940
1079
1145
1344
1415
1607
1733
```

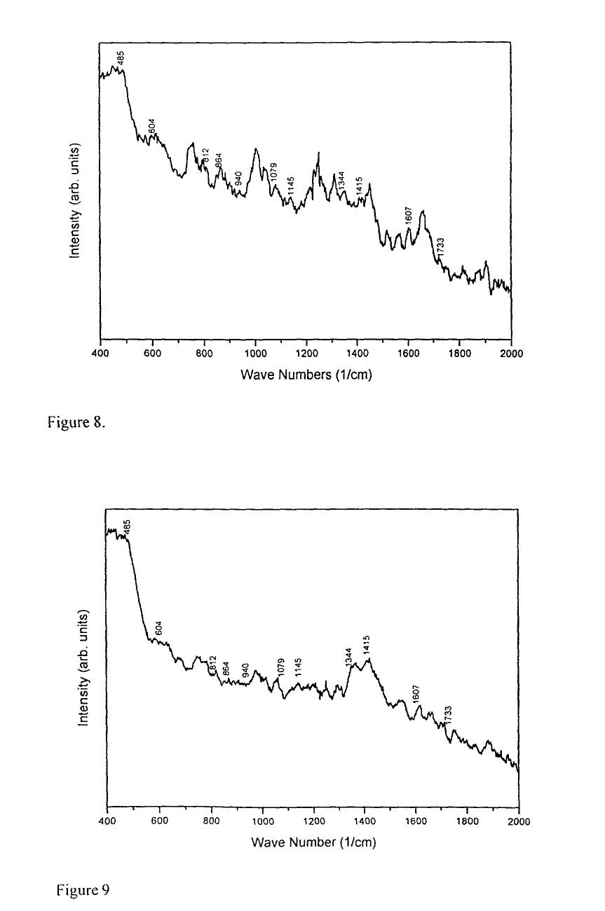

In this method, Raman spectra were acquired from ex vivo porcine whole eyes with MSG injected in the eyes to simulate disease conditions (9). Raman spectra were acquired with the optical system described above and shown in FIG. 4. The position of the porcine eye was varied relative to the focusing lens in order to demonstrate the effectiveness of this geometry for isolating signals from the vitreous. For the laser focusing on the ocular lens, it is expected that the strong Raman signal from the lens and cornea will mask the MSG signal. This spectrum is shown in FIG. 8, in which the MSG signal cannot be clearly observed. In this described apparatus, when the position of the eye is adjusted such that the laser focuses in the vitreous, it is expected that the Raman signals from the lens and cornea will be significantly reduced. The Raman spectrum for this alignment is shown in FIG. 9. As can be seen in FIG. 9, the Raman from the lens and cornea is not observed, and the Raman emission from MSG at 1344 and 1416 CM⁻¹ is clearly identifiable, although a background signal is also observed. This background is a combination of scattered laser light and low level fluorescence from the eye and optical components.

It should be understood that this invention can be extended to detect other biomolecules located in the vitreous and is not limited to detection of MSG.

### Data Analysis and Signal Processing Algorithms

The resultant Raman signal intensity is preferably analyzed via a quantifying system and compared to calibrated, chemically measured MSG standards. Other quantifying methods For calibrating Raman signal intensity would also be within the scope of the present invention.

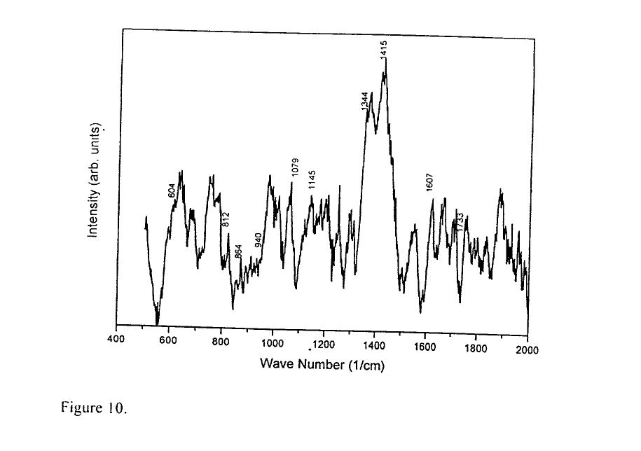

Signal processing algorithms can be used to enhance the MSG signal in order to improve the accuracy and sensitivity of the Raman measurements. These algorithms can include: data smoothing; Fourier filtering; background fluorescence wing subtraction; differentiation; integration; and differences between spectrum; as well as other algorithms. An example of one of these algorithms is applied to the spectra shown in FIG. 9 to reduce the background fluorescence signal. In this algorithm, this spectrum is fitted to a low order polynomial, using a least squares method. The polynomial is then subtracted from the original data. In the resulting spectrum, the background signals are greatly reduced, and the Raman lines are preserved as seen in the spectrum shown in FIG. 10, which is the Raman spectrum shown in FIG. 9 with the background subtracted.

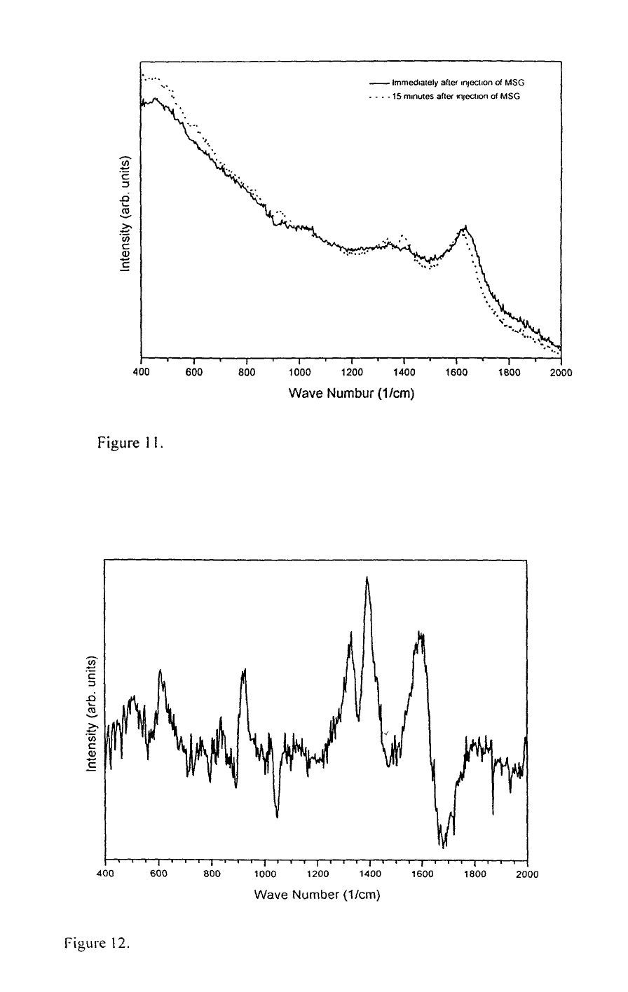

A demonstration of use of spectral differences t detect changes in MSG concentrations can be seen by examining the spectra shown in FIG. 11. This figure shows the Raman spectra acquired from a porcine eye cup immediately after MSG injection and 15 minutes after injection. The MSG was not injected directly in the path of the excitation laser but required several minutes to diffuse through the vitreous. Therefore, the spectrum acquired immediately afer injection interrogated by a region of the vitreous with little or no MSG at 1=0 but by 1=15 minutes, the concentration of MSG in the excited region of the vitreous increased significantly. For the two spectra shown in FIG. 11, the different spectra was calculated b first subtracting the background from each curve and then taking the differences between the curves. The background was subtracted by fitting each spectrum with a polynomial and then subtracting, as described above. The difference is shown in FIG. 12.

Although the invention is disclosed with reference to particular embodiments thereof, it will become apparent to those skilled in the art that numerous modifications and variations can be made which will fall within the scope and spirit of the invention as defined by the attached claims.

What is claimed is:

1. A system for performing in situ examination of the eye of a patient for detecting the presence and changes in biomolecular indicators in the vitreous humor, which are evidence of various eye diseases, said system comprising a monochromatic light source directing a monochromatic light beam through the front of the eye onto the vitreous humor a detector for detecting, scattered light of the laser light beam, a spectroscope for measuring by Raman spectroscopy, shift of the scattered light due to the presence of any indicators in the vitreous humor which are evidence of corresponding eye diseases, a diagnostic unit for correlating the shift of the scattered light to determine the eye disease, the system being constructed and arranged to minimize noise interference produced outside the vitreous humor.

2. The system as claimed in claim 1, wherein said monochromatic light source comprises a laser generator.

3. The system as claimed in claim 2, further comprising a wavelength selective optical device to isolate and detect the Raman signals.

4. The system as claimed in claim 3, wherein said detector comprises a photodetector for converting light signals to electrical signals.

5. The system as claimed in claim 3, comprising a lens system for expanding said light beam and focusing it into the vitreous humor to minimize said noise interference.

6. The system as claimed in claim 1, comprising optical fibers for delivering the monochromatic light beam to the eye and for collecting the Raman scattered light.

7. The system as claimed in claim 6, comprising filters in the light beam delivering fibers to eliminate fluorescence and Raman light generated in the fibers.

8. The system as claimed in claim 7, comprising further filters in the collecting fibers to eliminate excitation light and other background light sources.

9. The system as claimed in claim 1, wherein to minimize noise interference produced as Raman signals in the lens and cornea, said system comprises a lens system for expanding the monochromatic light beam at the lens and focusing the light beam in the vitreous humor.

10. The system as claimed in claim 1, further comprising a monitoring system for monitoring changes in Raman spectra from the patient over time to detect onset of disease.

11. The system as claimed in claim 1, further comprising a monitoring system for monitoring changes in Raman spectra from the patient over time to measure progression of disease.

12. The system as claimed in claim 11, further comprising a device to compare and calculate differences in Raman spectra between a normal eye and the eye of the subject patient.

13. The system as claimed in claim 12, wherein said device which compares and calculates differences locates and identifies these differences to track changes in the biomolecules.

14. The system as claimed in claim 1, wherein said detector includes signal processing algorithms to enhance measured changes.

15. The system as claimed in claim 1, wherein said diagnostic unit includes curve integration and differentiation means to enhance detection of disease.

16. The system as claimed in claim 1, wherein said diagnostic unit includes a system of background subtraction by polynomial filtering to enhance identification of disease.

17. The system as claimed in claim 1, wherein said diagnostic unit includes a device to carry out Fourier analysis to determine differences in spectra to enhance identification of disease.

18. The system as claimed in claim 1, wherein said diagnostic unit includes a device to carry out Fourier filtering to determine differences in spectra to enhance identification of disease.

19. The system as claimed in claim 1, wherein said detector is constructed to detect MSG.

20. The system as claimed 1, wherein said detector is constructed to detect ascorbic acid.
